(19) United States
(12) Patent Application Publication
(10) Pub. No.: US 2009/0305276 A1
(43) Pub. Date: Dec. 10, 2009

(54) Method for Predicting Skin Sensitizing Activity of Compounds

(76) Inventor: James M. McKim, Kalamazoo, MI (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/399,826

(22) Filed: Mar. 6, 2009

Related U.S. Application Data
(60) Provisional application No. 61/058,826, filed on Jun. 4, 2008.

(51) Int. Cl. C12Q 1/68; G01N 33/68

(52) U.S. Cl. 435/6; 436/86

(57) ABSTRACT

The present invention provides methods for predicting the in vivo skin sensitizing activity of chemical compounds using a combination of mammalian cell models with multiple endpoint analysis, time and concentration response curves. The methods allow the determination of a predicted in vivo sensitization value of a compound—for example, a EC3 LLNA value, a GPMT value or a IVTI value—without the use of animals, with a high degree of accuracy. The methods involve detecting expression levels of genes implicated in skin sensitization, combining expression level data with concentration response data, conducting a computational analysis, and comparing test compound data to a database of known skin sensitizers.

### FIELD OF THE INVENTION

The invention relates generally to in vitro methods for evaluating the likelihood that a compound will act as a skin sensitizer in vivo.

### BACKGROUND OF THE INVENTION

The chemical, pharmaceutical, cosmetic and personal care industries are required to identify hazards and evaluate potential risks to consumers of new drugs and personal care products. Additionally, recent legislation has limited or completely banned the use of animals for safety testing of products classified as cosmetics. As a result, there is a growing need for in vitro alternatives to standard animal safety tests.

The Globally Harmonized System for Classification and Labeling of Chemicals (GHS) defines a “skin sensitizer” as “a substance that will induce an allergic response following skin contact.” A substance is classified as a skin sensitizer when human data show that it can induce a sensitization response following skin contact in a substantial number of persons or when there are positive results from an appropriate animal test. Therefore, the prediction of the sensitizing capacity of a chemical is of importance to the chemical, pharmaceutical, cosmetic and personal care industries.

Currently, the most widely used methods in the art for prediction of sensitizing capacity still involve animal testing. In animals the process of chemical sensitization involves two distinct phases, induction and elicitation. (FIG. 1). Alternative Toxicological Methods, Salem, Ed H., Katz, SA., CRC Press (2003). During the induction phase or sensitization phase, the chemical allergen comes in contact with the animal's skin, and binds to proteins to form haptens. The Langerhan cells in the skin then take up and process the chemical-protein haptens. The Langerhan cells containing the haptens migrate to the lymph nodes and present the haptens to lymphocytes. Lymphocytes within the lymph node are then activated and cell proliferation rapidly and significantly increases; this is the hallmark of the induction phase. The second phase of chemical contact hypersensitivity is known as elicitation. Following cell proliferation in the lymph nodes, T-lymphocytes are considered “primed” and consequently possess a specific memory for the sensitizing agent. When exposed to the same chemical, an antigen-specific response occurs. The mechanism of induction is well known in the art and it is believed that most, if not all, chemical sensitizers possess intrinsic electrophilic properties or can be metabolized to intermediate metabolites with highly reactive electrophilic centers, which can interact with cellular nucleophiles such as glutathione.

A group of chemicals known as polycyclic aromatic hydrocarbons (PAHs), can be metabolized to reactive intermediates, which produce cell injury via increases in reactive oxygen species and interaction with cellular macromolecules. Ashby et al., “Mechanistic Relationship among Mutagenicity Skin Sensitization, and Skin Carcinogenicity,” Env. Health Perspectives, 101(1), 62-67 (1993). Studies designed to identify cellular defense mechanisms to redox stress identified a group of stress-response proteins whose intracellular levels can be induced by electrophiles and reactive oxygen via cytosolic transcription factors that bind to a control segment of DNA termed the antioxidant response element (ARE). Natsch and Emter, “Skin Sensitizers Induce Antioxidant Response Element Dependent Genes: Application to the in vitro Testing of the Sensitization Potential of Chemicals,” Toxicological Sciences, 102(1), 110-119, (2008). ARE is also known as the electrophilic response element or Eph/ARE. ARE/EphARE controls the expression of several genes capable of protecting the cell from reactive oxygen and electrophilic damage. The link between the chemical/reactive products and the expression of ARE controlled genes is a transcription factor known as NF-E2 p45-related factor 2 (Nrf2). This intrinsic sensor system detects reactive chemicals and reactive oxygen species and increases the expression of proteins, such as NAD(P)H quinone oxidoreductase 1 (NQO1), aldo-keto reductase (AKR), glutathione S-transferase (GST), and several other genes that protect cells from damage (Wang et al., 2004). An increase in the expression of these genes can be used as an indicator of reactive chemicals or their metabolites. (FIG. 2).

The murine local lymph node assay (LLNA) is a widely used predictive method for the identification and potency assessment of contact allergens, i.e., sensitizing capacity. Kimber et al. (1995) “An international evaluation of the murine local lymph node assay and comparison of modified procedures,” Toxicology 103:6373; Ashby et al., “Structure activity relationships in skin sensitization using murine local lymph node assay,” Toxicology, 103, 177-194 (1995). The LLNA is based on a correlation between the vigor of a proliferative response induced in the local draining lymph nodes by topically applied chemicals and the extent of sensitization developed. Lymphocyte activation within the lymph node, which leads to the induction phase, is the mechanism underlying the LLNA. The results of the LLNA are expressed as a stimulation index (SI), which is the proliferative response ratio between test and control groups. Test materials that at one or more concentrations cause an SI of 3 or higher are considered to be positive in the LLNA, and calculated EC3 values (the estimated concentration required to produce an SI of 3) are used to compare the sensitizing capacity of different test materials. Basketter et al. (1999) “A comparison of statistical approaches to the derivation of EC3 values from local lymph node assay dose responses” J. Appl. Toxicol. 19:261-266.

Other predictive methods include the guinea pig maximization test, GPMT, and the Buehler test in guinea pigs. Andersen and Maibach (1985) “Contact Allergy Predictive Test in Guinea Pigs,” Vol. 14, Karger, Basel. See Karlberg et al. (2008) “Allergic Contact Dermatitis—Formation, Structural Requirements, and Reactivity of Skin Sensitizers,” Chem. Res. Toxicol. 21:53-69. It has also been shown that chemically reactive compounds or compounds metabolized to reactive intermediates can also cause chemical sensitization. Several PAHs (e.g., menadione and benzo(a)pyrene) are also classified as sensitizers.

Gerberick et al. described an in vitro method of identifying chemicals that are highly reactive, and as such possess electrophilic centers with a cell free system. Gerberick et al., “A chemical dataset for evaluation of alternative approaches to skin-sensitization testing,” Contact Dermatitis, Vol. 50, 274-288 (2004). This approach utilized three peptides with nucleophilic sites and measured the binding of the test agent to the peptide as a measure of reactivity. The method identified chemical sensitizers based on their ability to bind to peptides in a cell free system. The system correctly identified true negatives with a higher degree of confidence than true positives. The system did not however provide a prediction of in vivo or animal responses, such as the LLNA EC3 value or provide an estimated no effect level (NOEL) for human exposure. In addition, the high degree of error for correctly identifying true positives made the approach not a viable stand alone assay for identifying chemical sensitizers.

Natsch and Emter (2008) described the use of the engineered cell line (AREc32 cells) derived from human breast (MCF-7) cells (Wang et al., 2006) to identify chemical sensitizers by monitoring the expression of a reporter gene linked to the ARE promoter. The AREc32 system was used to evaluate more than 100 chemicals with varying degrees of potency for sensitization. This system identified chemicals as positive or negative, but failed to provide a predictive link to in vivo data and did not differentiate the major classes of sensitizers: weak, moderate, strong, and extreme. One disadvantage is that the cell system was derived from human breast cancer and not from human skin, thus this system is not representative of the target tissue. Another disadvantage of this system is the need to culture the test cells in monolayer covered with culture medium. This means that compounds with high logP or low water solubility cannot be tested in this model.

The ability to reliably assess compounds likely to be classified as skin sensitizers without the use of animals has not been described. Moreover, the ability to extrapolate in vitro data into a predicted in vivo exposure level that would produce toxicity in the skin has not been developed. Thus, in vitro cell-based models able to predict toxicity specific to the skin would be of considerable value in early drug, cosmetic, and other product development. The present invention provides a novel non-animal test for skin sensitization and respiratory sensitization.

### SUMMARY OF THE INVENTION

The invention provides methods for predicting the in vivo skin sensitizing activity of a test compound.

In a first main embodiment, the invention provides a method for predicting the in vivo skin sensitizing activity of a compound, comprising: (a) culturing mammalian cells; (b) applying a concentration of a test compound to the cells of step (a); (c) measuring the expression level of one or more marker genes in the cells of step (b); (d) optionally monitoring multiple endpoints of cell viability and general cell health; (e) conducting a computational analysis of the concentration applied in step (b) and the expression level(s) measured in step (c); and (f) determining a predicted in vivo sensitization value based on the analysis of step (e).

In a further embodiment, the invention provides a method further comprising plotting the value calculated in step (e) against one or more known LLNA EC3 values and determining a predicted LLNA EC3 value.

In another embodiment, the mammalian cells are selected from the group consisting of: human keratinocytes (HaCat cells), 3D human skin cells, normal human epithelial cells (NHEK cells), MCF7 cells, H4IIE cells, and combination cultures including keratinocyte and dendritic cells.

In yet another embodiment, the test compound is applied to the cells in varying dosage amounts.

In certain embodiments, the marker gene(s) are selected from the group consisting of: quinone reductase, IL-8, ALDHc, CYP1A, GCLA, GST, HO1, MafF, NQO1, hMTT, GAPDH CD-86, AKR, TXN, and TXN reductase.

The in vivo sensitization value may be any appropriate value, including but not limited to a LLNA EC3 value; a GPMT value; and a IVTI value.

In another embodiment, the method described above further comprises: (i) incubating the test compound with a polypeptide to allow for binding; and (ii) measuring the amount of unbound polypeptide; prior to the step of culturing mammalian cells, as described above. In certain embodiments of this method, the polypeptide is GSH. In other embodiments, the test compound of step (i) is applied in varying dosage amounts. In still other embodiments, the method further comprises adding human liver microsomal protein in step (i).

In a second main embodiment, the invention provides a method for determining the sensitizing activity of a compound comprising: (a) incubating a test compound with a polypeptide to allow for binding; and (b) measuring the amount of unbound polypeptide. In certain embodiments of this method, the polypeptide is GSH. In other embodiments, the test compound is applied in varying dosage amounts. In still other embodiments, the method further comprises adding human liver microsomal protein in step (a).

### BRIEF DESCRIPTION OF THE DRAWINGS

FIG. 1 shows the multiple-step process of sensitization.

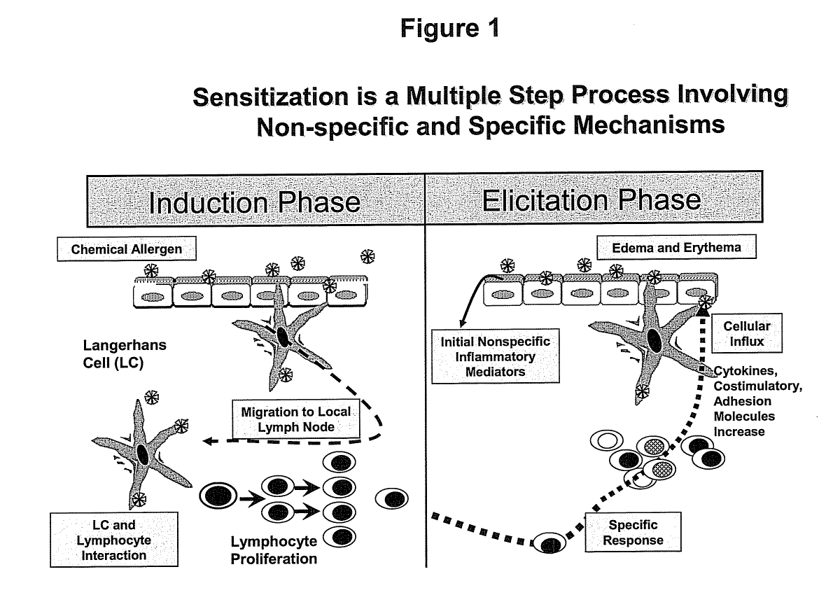

FIG. 2 shows the intrinsic sensor system and detection of electrophiles.

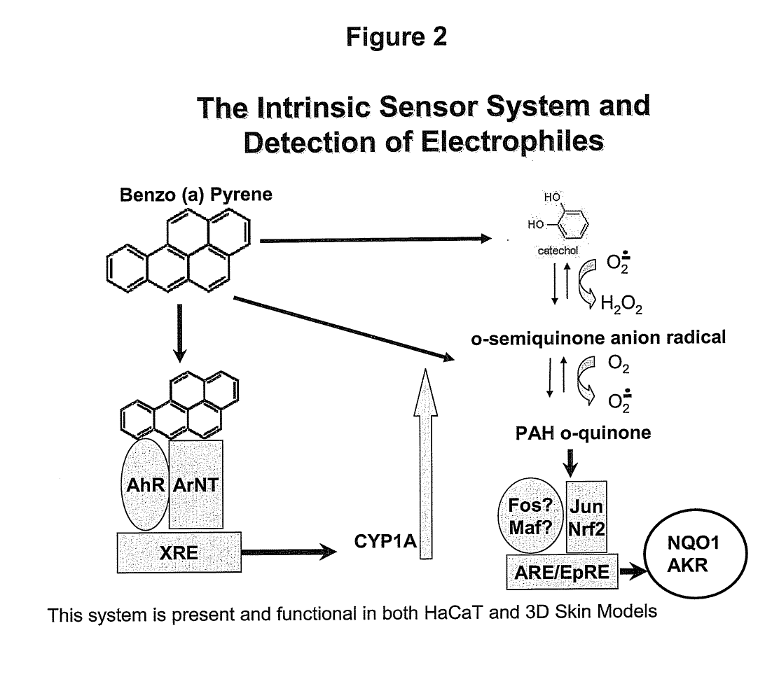

FIG. 3 shows the calibration curve for the GSH assay. The liner range of the assay stops at concentration of GSH above 10 μM.

FIG. 4 shows levels of GSH-binding for non-sensitizers and extreme sensitizers.

FIG. 5 shows EC3 LLNA values plotted against in vitro tox index values. The linear function is shown inset.

FIG. 6 shows evaluation of sensitization strength of various compounds.

FIG. 7 shows exponential regression analysis of IVTI plotted against known in vivo LLNA EC3 data.

FIG. 8 shows correlation between predicted LLNA EC3 values and known LLNA EC3 values for humans and guinea pig. The known values are taken from The Research Institute for Fragrance Materials, Inc.

### DETAILED DESCRIPTION

The following definitions are used throughout the present disclosure: “LLNA” is an abbreviation for the murine local lymph node assay; “LLNA EC3 value” is an abbreviation for the estimated concentration required to produce a stimulation index of 3 in the LLNA; “NOEL” is an abbreviation for No Effect Level, i.e., the highest concentration of the chemical compound at which a measurable toxic effect of the chemical compound is not observable; IC₅₀ is an abbreviation for a measure of the effectiveness of a compound in inhibiting biological or biochemical function, i.e. how much of a particular drug or other substance (inhibitor) is needed to inhibit a given biological process (or component of a process, i.e. an enzyme, cell, cell receptor or microorganism) by half, IC₉₀ is an abbreviation for a measure of the effectiveness of a compound in inhibiting biological or biochemical function, i.e. how much of a particular drug or other substance (inhibitor) is needed to inhibit a given biological process (or component of a process, i.e. an enzyme, cell, cell receptor or microorganism) by 90%; “DMSO” is an abbreviation for dimethylsulfoxide; “IL-8” is an abbreviation for Interleukin-8, a chemokine produced by macrophages and other cell types such as epithelial cells; “CD-86” is an abbreviation for a gene encoding a type I membrane protein.

Relevant background information is available in U.S. Pat. No. 6,998,249 issued to McKim and Cockerell on Feb. 14, 2006 and entitled “TOXICITY SCREENING METHOD” and U.S. Appl. No. 20070218457 submitted by McKim on Mar. 6, 2007 and entitled “TOXICITY SCREENING METHODS”, the contents of which are expressly incorporated into this disclosure in their entirety.

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary and not exhaustive. Also, it is to be understood that the phraseology and terminology employed herein is for the purpose of description and should not be regarded as limiting.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al., Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The present invention provides methods for predicting the in vivo skin sensitizing activity of chemical compounds using a combination of mammalian cell models with multiple endpoint analysis, time and concentration response curves. In particular, the present invention provides in vitro screening methods by detecting the expression of gene markers associated with skin sensitization. In preferred embodiments, these methods involve assays to measure the expression of key genes associated with skin sensitization in multiple mammalian cell models, coupled with multiple endpoint analysis, time, and concentration response curves. These methods can provide a means of predicting the potential of a chemical to act as a skin sensitizer. The genes associated with skin sensitization include but are not limited to quinone reductase, IL-8, CD-86, aldo-keto reductase, thioredoxin, and thioredoxin reductase.

The use of some of these genes as markers for skin sensitization is not novel and has been reported in the literature. However, the use of these markers in multiple mammalian cell models, combined with concentration response, and computational analysis to provide an in vivo toxicity value has not been reported. The accuracy of the in vitro prediction of in vivo toxicity is improved by using training set compounds of varying potencies. The process of analyzing the in vitro data to provide an estimated risk factor for skin sensitization involves the analysis of the concentration response curves and the magnitude of expression. A combination of data that encompass the no effect level (NOEL), IC₅₀, and IC₉₀ points on the concentration response curve provide key reference values for estimating the in vivo exposure and for categorizing the severity of the chemical response. The methods allow the determination of a predicted in vivo sensitization value—for example, the LLNA EC3 value or GPMT value. Furthermore, a database of known skin sensitizers is used to compare the effects of unknown chemicals and to provide important perspective with regard to predicting in vivo toxicity.

### Prediction of In Vivo Skin Sensitization

Specifically, chemicals would be placed in an appropriate vehicle such as DMSO and applied to appropriate mammalian cells, such as human keratinocytes (HaCat cells), 3D human skin cells, normal human epithelial cells (NHEK cells), MCF7 cells, H4IIE cells (ATCC Accession #CRL-1548), or combination cultures including keratinocyte and dendritic cells. Following exposure times of 6, 24, and 72 hr the expression levels of several genes that are associated with an increase risk for sensitization type reactions. The expression of key gene markers associated with sensitization that include but are not limited to quinone reductase, IL-8, CD-86, aldo-keto reductase, thioredoxin, and thioredoxin reductase is measured by RT-PCR.

In particular aspects, these assays will involve culturing human keratinocytes (HaCat cells), 3D human skin cells, normal human epithelial cells (NHEK cells), MCF7 cells, H4IIE cells or combination cultures with keratinocyte and dendritic cells in culture medium that comprises a plurality of concentrations of the chemical compound; measuring the expression level of one or more gene markers associated with skin sensitization in response to culturing in at least three concentrations of the chemical compound over at least three different time points and predicting the EC3 LLNA value, GPMT value, no effect level (NOEL), IC₅₀, and IC₉₀ of the chemical compound from such measurements. The various embodiments involved in conducting such assays are described in further detail below.

The test compounds might be received pre-weighed into glass vials. The preparation of 20 millimolar stock solutions will be accomplished by adding a sufficient amount of dimethylsulfoxide (DMSO) directly into the vials. These stocks will be used to prepare 200 micromolar stock solutions in DMSO. Both the 20 millimolar and 200 micromolar, stocks will be used to prepare dosing solutions of 0.05, 0.1, 1.0, 5.0, 10.0, 20.0, 50.0, 100, and 300 micromolar in culture media with a final DMSO concentration of 0.5%. The stocks and dosing solutions will be prepared on the day prior to dosing. The solutions will be wrapped in foil and stored at 4° C. until the next morning.

Negative and positive controls are included with every assay. Assay response is continually monitored to assure reliable results. Camptothecin and rotenone might be included as positive controls for all endpoints, while DMSO at 0.5% in culture medium with and without cells might be included as a negative control.

The foregoing method requires preparing cell cultures. Such a cell may be a primary cell in culture or it may be a cell line. The cells may be obtained from any mammalian source that is amenable to primary culture and/or adaptation into cell lines. In lieu of generating cell lines from animals, such cell lines may be obtained from, for example, American Type Culture Collection, (ATCC, Rockville, Md.), or any other Budapest treaty or other biological depository.

The cells used in the assays preferably derived from tissue obtained from humans. Techniques employed in mammalian primary cell culture and cell line cultures are well known to those of skill in that art. Indeed, in the case of commercially available cell lines, such cell lines are generally sold accompanied by specific directions of growth, media and conditions that are preferred for that given cell line.

Once the cell cultures are thus established, various concentrations of the chemical compound being tested are added to each cell media and the cells are allowed to grow exposed to the various concentrations of a test chemical compound for 6, 24, and 72 hours. Furthermore, the cells may be exposed to the test chemical compound at any given phase in the growth cycle. For example, in some embodiments, it may be desirable to contact the human keratinocytes (HaCat cells), 3D human skin cells, normal human epithelial cells (NHEK cells), MCF7 cells, H4IIE cells, or combination cultures with keratinocyte and dendritic cells with the compound at the same time as a new cell culture is initiated. Alternatively, it may be desirable to add the compound when the cells have reached confluent growth or arc in log growth phase. Determining the particular growth phase that cells are in is achieved through methods well known to those of skill in the art.

The varying concentrations of the given test compound are selected with the goal of including some concentrations at which no toxic effect is observed and also at least two or more higher concentrations at which a toxic effect is observed. A further consideration is to run the assays at concentrations of a compound that can be achieved in vivo. For example, assaying several concentrations within the range from 0 micromolar to about 300 micromolar is commonly useful to achieve these goals. It will be possible or even desirable to conduct certain of these assays at concentrations higher than 300 micromolar, such as, for example, 350 micromolar, 400 micromolar, 450 micromolar, 500 micromolar, 600 micromolar, 700 micromolar, 800 micromolar, 900 micromolar, or even at millimolar concentrations. The estimated therapeutically effective concentration of a compound provides initial guidance as to upper ranges of concentrations to test. For certain chemicals, the maximum soluble concentration may be used to establish the highest exposure concentration.

In an exemplary set of assays, the test compound concentration range under which the assay is conducted comprises dosing solutions which yield final growth media concentration of 0.05 micromolar, 0.1 micromolar, 1.0 micromolar, 5.0 micromolar, 10.0 micromolar, 20.0 micromolar, 50.0 micromolar, 100 micromolar, and 300 micromolar of the compound in culture media. As mentioned, these are exemplary ranges, and it is envisioned that any given assay will be run in at least two different concentrations, and the concentration dosing may comprise, for example, 4, 5, 6, 7, 8, 9, 10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 80.0 micromolar, 10.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 210.0 micromolar, 220.0 micromolar, 230.0 micromolar, 240.0 micromolar, 250.0 micromolar, 260.0 micromolar, 270.0 micromolar, 280.0 micromolar, 290.0 micromolar, and 300 micromolar in culture media. It will be apparent that a cost-benefit balancing exists in which the testing of more concentrations over the desired range provides additional information, but at additional cost, due to the increased number of cell cultures, assay reagents, and time required. In one embodiment, ten different concentrations over the range of 0 micromolar to 300 micromolar are screened.

Typically, the various assays described in the present specification may employ human keratinocytes (HaCat cells), 3D human skin cells, normal human epithelial cells (NHEK cells), MCF7 cells, H4IIE cells, or combination cultures with keratinocyte and dendritic cells seeded in 96 well plates or 384 cell plates. The cells are then each exposed to the test compounds over a concentration range, for example, 0-300 micromolar. The cells are incubated in these concentrations for a given period of, for example, 6, 24, and 72 hours. In one embodiment, all the assays are performed in at least triplicates at the same time such that a complete set of data are generated under similar conditions of culture, time and handling. However, it may be that the assays are performed in batches within a few days of each other.

### Measuring the Expression of Gene Markers

In specific embodiments, the expression of gene markers is measured. Appropriate gene markers for the methods of the invention include but are not limited to quinone reductase, IL-8, CD-86, aldo-keto reductase, thioredoxin, and thioredoxin reductase. Expression levels may be measured by any appropriate method of measuring gene expression, including but not limited to polymerase chain reaction (PCR), reverse transcriptase PCR (RT-PCR), fluorescent in situ hybridization (FISH), branched DNA (bDNA) assay, differential display, RNA interference, reporter genes, microarrays, and proteomics. For instance, expression levels may be measured by RT-PCR in at least triplicates. The standard protocols for performing RT-PCR may be obtained from Invitrogen, (Carlsbad, Calif.). Standard methods and protocols for measuring gene expression are described in the literature and in treatises such as Sambrook et al., “Molecular Cloning: A Laboratory Manual” and Avison, Matthew, “Measuring Gene Expression,” Taylor and Francis (2006). These protocols are incorporated herein by reference in their entirety.

### Monitoring General Cell Health

In specific embodiments, general cell health may be monitored. The indicators of cell health and viability include but are not limited to, indicators of cellular replication, mitochondrial function, energy balance, membrane integrity and cell mortality. In other embodiments, the indicators of cell health and viability further include indicators of oxidative stress, metabolic activation, metabolic stability, enzyme induction, enzyme inhibition, and interaction with cell membrane transporters.

The specific assay to monitor any of the given parameters is not considered crucial so long as that assay is considered by those of skill in the art to provide an appropriate indication of the particular biochemical or molecular biological endpoint to be determined, such as information about mitochondrial function, energy balance, membrane integrity, cell replication, and the like. The following sections provide exemplary assays that may be used in the context of the present invention.

The ability of cells to divide requires coordinated signaling between a vast array of intracellular receptors. Cell replication or “mitogenesis” requires the cells to be functioning at optimum. A change in the ability to replicate is therefore an indication of stress or abnormal function. An exemplary assay that will allow the determination of cell replication is the CYQUANT® assay system from Invitrogen, Molecular Probes (Carlsbad, Calif.). Additional assays that may be used to provide an indication of mitogenesis may include, but are not limited to, monitoring ³H-thymidine incorporation and a BrdU incorporation assay. In addition, mitogenesis may be monitored by determining the function, presence or absence of a component that controls cell cycle. Exemplary components will be well known to those of skill in the art and include, but are not limited to, p53, p21, TGF-.beta., CDK1, PCNA and the like.

Compounds that produce direct effects on the cells typically alter mitochondrial function, by either up- or down regulating oxidative respiration. This means that cellular energy in the form of ATP may be altered. Mitochondrial function can be used as an indicator of cytotoxicity and cell proliferation. Healthy mitochondria catalyze the reduction of 3-(4,5-dimethylthiazol-2-yl)-2,5-diphenyltetrazolium bromide (MTT) to a blue or purple formazan compound. The relatively insoluble formazan blue is extracted into isopropanol and the absorbance of the extract measured. A high absorbance value indicates viable cells and functional mitochondria. Conversely, a decrease in the intensity of color suggests either a loss of cells, or direct toxic effects on the mitochondria. The MTT assay is well known to those of skill in the art and has been described in for example, the MTT mitochondrial dye assay is described in Mosmann, J. Immunol. Methods 65, 55-63, 1983 and in Denizot et al., J. Immunol. Methods. 89, 271-277, 1986. A similar assay that monitors XTT mitochondrial dye is described by Roehm et al., J. Immunol. Methods, 142, 257-265, 1991. In addition, those of skill in the art also may determine mitochondrial function by performing for example an Alamar Blue assay [Goegan et al., Toxicol. In vitro 9, 257-266. 1995], a Rhodamine 123 assay, or a cytochrome C oxidase assay.

ATP provides the primary energy source for many cellular processes and is required to sustain cell and tissue viability. Intracellular levels of ATP decrease rapidly during necrosis or apoptosis. Therefore, changes in the cellular concentration of ATP can be used as a general indicator of cell health. When normalized on a per cell basis, ATP can provide information on the energy status of the cell and may provide a marker to assess early changes in glycolytic or mitochondrial function. Assays that allow a determination of ADP/ATP energy balance are well known in the art (Kangas et al., Med. Biol., 62, 338-343, 1984).

Other assays for determining membrane integrity include, but are not limited to, assays that determine lactate dehydrogenase activity, aspartyl aminotransferase, alanine aminotransferase, isocitrate dehydrogenase, sorbitol dehydrogenase, glutamate dehydrogenase, ornithine carbamyl transferase, γ-glutamyl transferase, and alkaline phosphatase.

### Predicting In Vivo Skin Sensitizing Activity of a Compound From In Vitro Analyses

Once all data for expression of one or more gene markers in cultured mammalian cells in culture medium that comprises a plurality of concentrations of the chemical compound are received, the data are analyzed to obtain a detailed profile of the compound's toxicity. For example, most conveniently, the data are collated over a dose response range in a single cell line on a single graph. In such an embodiment, the measurement evaluated for each gene marker at any given concentration is plotted as a percentage of a control measurement obtained in the absence of the compound. However, it should be noted that the data need not be plotted on a single graph, so long as all the parameters are analyzed collectively to yield detailed information of the effects of the concentration of the compound on the different parameters to yield an overall toxicity profile. The final evaluation of gene expression changes should be assessed at exposure concentrations and at exposure times that allow for at least about 75% of the cells to remain viable. Higher concentrations or longer exposure times may decrease cell viability to a level where accurate assessment and evaluation of the marker genes cannot be determined.

In certain embodiments, the determination of a predicted in vivo sensitization value comprises performing concentration response analyses of measurements from at least three separate assays for each gene marker in each of the cell line. From these concentration response analyses, a combination of data that include the NOEL, IC₅₀ and IC₉₀ points on the concentration response curve provide key reference values for determining a predicted in vivo sensitization value (such as the EC3 LLNA value or GPMT value), for estimating the in vivo exposure, and for categorizing the severity of the chemical response.

### Determination of Sensitizing Strength

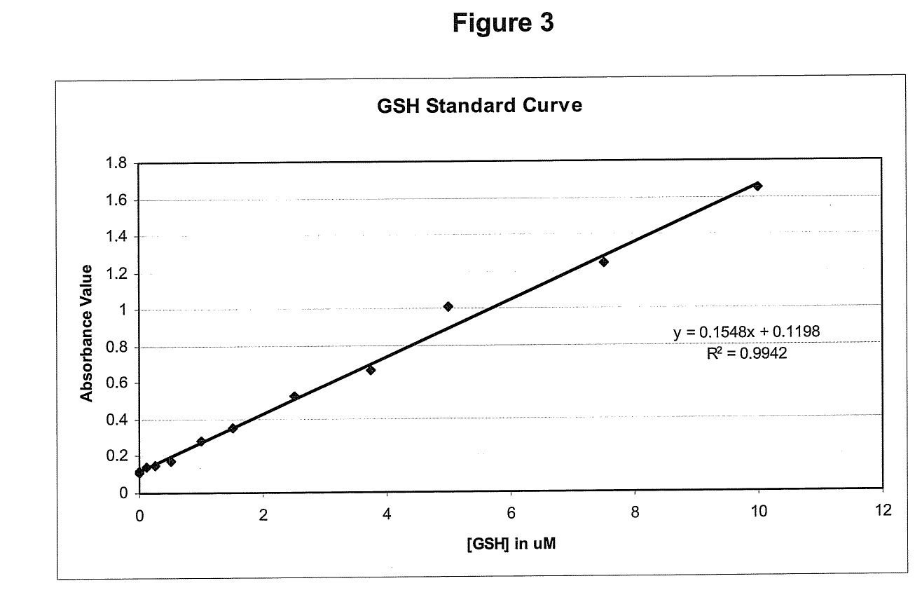

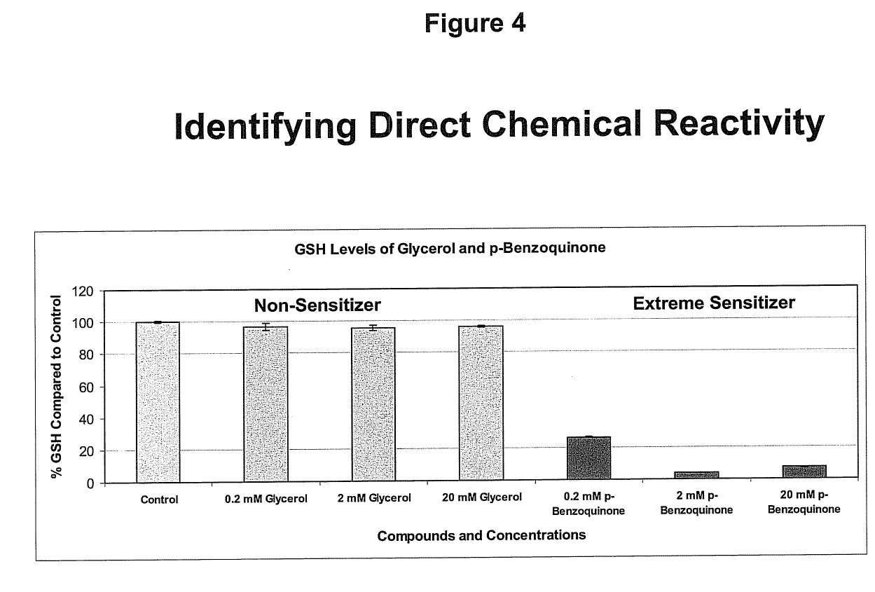

Direct and indirect chemical reactivity of a test compound can be determined using a glutathione depletion assay with and without metabolizing enzymes. Glutathione is a tripeptide consisting of three amino acids, glycine, glutamic acid, and cysteine, and is the principle antioxidant of cells. In one embodiment reduced glutathione (GSH) and the test compound are added in a ratio of 1:10, 1:100, 1:250, 1:500 1:750, or 1:1000. In a preferred embodiment, GSH and the test compound are added at a ratio of 1:100. Compounds that deplete more than about 80% of the GSH are considered to be strong sensitizers, and compounds that deplete less than about 10% are considered to be weak sensitizers. In one embodiment, the experiment can also be performed in the presence of 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1 mg/mL of microsomal protein from human liver in order to identify those chemicals that require metabolic conversion to reactive intermediates. In a preferred embodiment, 0.5 mg/mL of microsomal protein is added. An example of one chemical is dinitrochlorobenzene, which does not have a high degree of direct reactivity toward GSH, but is an extreme sensitizing agent. This compound undergoes metabolic conversion to a reactive intermediate that depletes GSH by covalently binding to it. Heylings, et al., “A prevalidation study on the in vitro skin irritation function test (SIFT) for prediction of acute skin irritation in vivo: results and evaluation of ECVAM Phase III”, Toxicology in Vitro 17:123-128 (2000). In a preferred embodiment, microsomal glutathione S-transferase (mGST) is used in combination with cytochrome P450. In another embodiment, this assay includes other polypeptides with sulfhydryl groups capable of reacting with electrophiles, either instead of or in addition to GSH. This prescreening step identifies strong and weak sensitizers, and establishes metabolic dependence (FIGS. 3 and 4).

### Prediction of LLNA EC3 Values

When predicting LLNA EC3 values and determining the sensitizing ability of compounds, analysis should be done using multiple parameters, i.e., multiple genes, toxicity, solubility, chemical reactivity. Use of more than just a single approach will ensure the high degree of accuracy needed for accurate prediction.

Human immortalized keratinocytes (HaCaT) are used for compounds that demonstrate good water solubility in order to test for dermal sensitizers in a species and organ specific model, For compounds with low solubility, mixtures, or finished products a human reconstructed epidermal model (EpiDerm, MatTek or EpiSkin, SkinEthic) is used. All of these cell systems possess the CYP1A enzyme and the Nrf2 mediated ARE/EpARE pathway. Cell viability may be determined using any accepted assay. Examples include, but are not limited to, MTT and lactate deghydrogenase (LDH). Normalization of gene expression is important to the development of reliable and reproducible data. Most RNA or gene expression methods use a single house keeping gene such as GAPDH or beta actin. However, several groups have shown that the use of a single gene for normalization leads to erroneous conclusions. Therefore, a minimum of four genes may be used to correctly determine chemical sensitization. The identity of the four genes used is dependent on expression levels and tissue type. For example, the HaCaT cell line is different than the human reconstructed epidermis models. Metallothionien is induced by metals and by oxidative stress. The inclusion of this gene allows unknown test compounds to be identifies as a metal sensitizer.

Eleven target genes are monitored and are shown in Table 1. All genes except metallothionein, GAPDH, and IL-8 are controlled by Nrf2 and ARE/EpARE. These genes serve as intrinsic sensors of reactive and electrophilic compounds.

TABLE 1

| Gene | Description |
|---|---|
| AKR | Aldoketo Reductase |
| ALDHc | Cytosolic Aldehyde Dehyrogenase |
| CYP1A | Cytochrome P450 1A |
| GAPDH | Glyceraldehyde-3-Phosphate Dehydrogenase |
| GCLC | Gamma Glutamyl Cysteine Synthetase |
| GST | Glutathione S Transferase |
| HO1 | Heme Oxygenase 1 |
| IL-8 | Interluekin 8 |
| MafF | small Maf leucine zipper transcription |
| | factors association with EpRE |
| NQO1 | NADPH Quinone Reductase |
| TXN | Thioredoxin |
| hMTT | Metallothionein |

Of particular value to the present invention is IL-8, which is a stress induced cytokine that provides an indication of cellular stress that may not be linked to sensitization. The inclusion of IL-8 and other cytokines not linked to Nrf2 and ARE provide a means of identifying potential irritants. Other examples of genetic markers for irritants include IL-1, IL-6, TNF alpha. Inclusion of these genetic markers makes it possible to differentiate chemicals that cause irritation but not sensitization.

The first analysis step following the assay is determining the in vitro toxicity index (IVTI). IVTI is an algorithm that examines viability, reactivity, number of genes that are increased more than 2.5 fold over controls, and the magnitude of induction for each gene. The IVTI value is an index assigned by an algorithm based on a series of queries. Viability should be greater than about 50% at any exposure concentration to allow for gene expression data at that concentration to be evaluated. The number of genes that are induced to a level greater than 2.5 fold are then determined. A value of 1 is assigned for each gene that responds. For example, if a compound induced three of the four genes to a degree greater than 2.5 a value of 3 would be assigned.

The next evaluation step concerns potency or the lowest concentration where a greater than 2.5 fold induction is observed. If the observation occurs at or below a concentration of 5 μM, another 1 is assigned and added to the previous assignment. If the gene with the lowest exposure concentration responded at a concentration greater than 500 μM, then a 1 is subtracted from the total. If the reactivity is high based on GSH depletion (greater than about 80% depletion) another 1 is added. If, on the other hand, there is less than about 10% reduction in GSH, then 2 points are subtracted.

When the IVTI is high the sensitization response is more potent and the assigned category becomes more severe. When the IVTI values from a large number of known sensitizers are plotted against LLNA EC3 values the data fit an exponential regression line with a correlation of approximately 92%. The equation for the line to be generated from the in vitro data enables the LLNA value to be predicted. It is the combined use of multiple gene markers, viability, reactivity, and generation of an IVTI that allow the test system to provide positive identification of chemical sensitizers in multiple human skin cell models. This model may also be applied to respiratory cell models, such as a human three-dimensional respiratory airway model to identify respiratory sensitizers. One example is the EpiAirway™ model by MatTek. This model may also be applied to skin human skin cell models, such as a human three-dimensional skin model. Examples are the EpiSkin™ by Skin Ethic and the EpiDerm™ by MatTek.

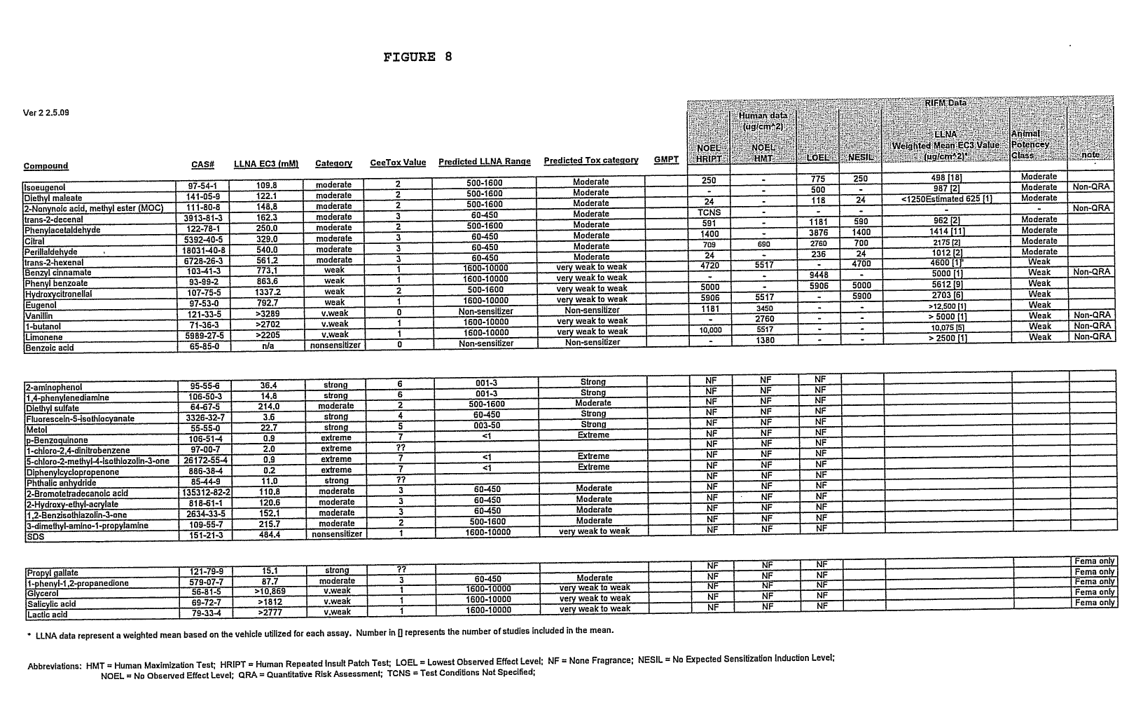

In addition to predicting animal LLNA EC3 values, the invention can be used to predict guinea pig GMPT, human repeat insult patch test (HRIPT) and HMT. Results of this correlation are shown in FIG. 8. In order for these correlations to be meaningful, data may be calculated using multiple exposure concentrations. The use of this analysis platform to determine IVTI coefficients allows for the combination of multiple data sets into a single value for sensitization.

The IVTI coefficients generated from this large data set, or training set, allow for conversion of the values to an in vivo estimate. The data are then used in an exponential regression equation to generate a response curve and therefore correlate with in vivo data (e.g. LLNA). This results in accurate prediction of the LLNA EC3, the GMPT, and the HRIPT values. This cross species extrapolation is novel in the art.

### Assay Kits

Another embodiment of the present invention provides a kit for practicing the methods described herein. In certain aspects of the present invention, all the necessary components for conducting the detection of expression of key genes associated with skin sensitization may be packaged into a kit. Specifically, the present invention provides a kit for use in a detection of expression of key genes associated with skin sensitization, the kit comprising a packaged set of reagents for conducting the detection by means of RT-PCR. The kits also may comprise the reagents for measuring and analyzing gene expression data that encompass the no effect level (NOEL), IC₅₀, and IC₉₀ points on the concentration response curve. The kits also may comprise other reagents for conducting additional detection and assays.

In addition to the reagents, the kit preferably also includes instructions packaged with the reagents for performing one or more variations of the detection assay of the invention using the reagents. The instructions may be fixed in any tangible medium, such as printed paper, or a computer readable magnetic or optical medium, or instructions to reference a remote computer data source such as a World Wide Web page accessible via the Internet.

### EXAMPLES

The present invention is further illustrated by the following examples, which should not be construed as limiting in any way. While some embodiments have been illustrated and described, it should be understood that changes and modifications can be made therein in accordance with ordinary skill in the art without departing from the invention in its broader aspects as defined in the following claims.

### Example 1

As shown in Table 2 (below), preliminary data were collected and used to build the algorithm for predicting the sensitization. NQO1 is quinone reductase; IL-8 is interleukin-8; and AKR is aldo-keto reductase.

TABLE 2

| | Sensitizer | LLNA | | | |
|---|---|---|---|---|---|
| Compound | Class | E3C | NQO1 | IL-8 | AKR |
| DMSO | | | | | |
| Phthalic Anhydride | Strong | 10.8 | ++ | + | ++ |
| Propyl Gallate | Strong | 15.09 | ++ | ++ | ++ |
| Isoeugenol | Moderate | 110 | + | + | ++ |
| Benzyl Cinnamate | Weak | 773 | ++ | −−− | ++ |
| Phenyl Benzoate | Weak | 863 | + | + | −−−− |
| Vanillin | Very Weak | >3000 | + | −−−− | −−−− |
| Benzoic Acid | Non- | | −−−− | −−−−− | −−−−− |
| | sensitizer | | | | |

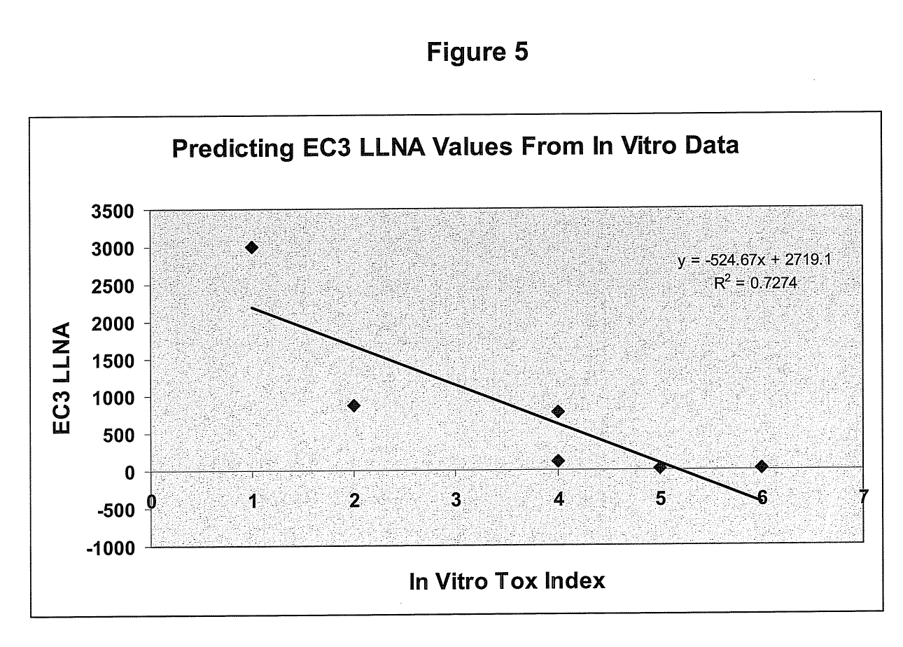

The compounds listed in Table 2 are known skin sensitizers with different magnitudes of effect. In this example, three gene markers are measured to develop a relationship between the magnitude of response, the number of genes responding above about 15%, and the minimum exposure concentration where expression occurred. The ++ signs indicate positive response and the number represents the magnitude of response. The dashed lines indicate no measurable response. Based on these data, an “in vitro sensitization index” (or, “in vitro tox index”) was determined and used to develop the regression curve shown in FIG. 5.

As shown in FIG. 5, a linear regression analysis provides excellent correlation and enables one to extrapolate or predict the LLNA value (in vivo effect) from the in vitro data (in FIG. 5, the “in vitro tox index”). The magnitude and frequency of gene response above about 15% is important to the extrapolation algorithm. The “in vitro tox index” (or “in vitro sensitization index”) can be plotted against known LLNA data, as shown in FIG. 5, or, alternatively, against known GPMT data.

### Example 2

Direct and indirect chemical reactivity of a test agent is determined using a glutathione depletion assay with and without metabolizing enzymes. A 100 mM stock of phosphate buffered saline (PBS) at pH 7.4 is used as the testing medium. Reduced glutathione (GSH) and the test compound are added in a ratio of 1:100. The reaction mixture is allowed to incubate at room temperature for 15 min. Following the incubation period the amount of free GSH or GSH not bound to test compound is measured. The same experiment is performed in the presence of 0.5 mg/mL microsomal protein from human liver in order to identify those chemicals that require metabolic conversion to reactive intermediates.

### Example 3

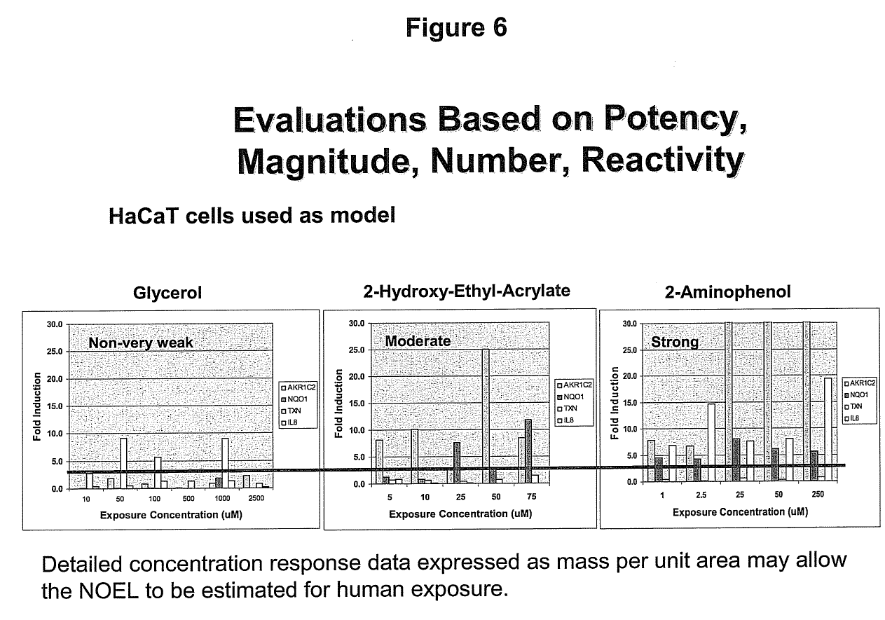

In order to test for dermal sensitizers in a species and organ specific model, human immortalized keratinocytes (HaCaT) are used for compounds that demonstrate good water solubility. The cells are cultured in standard media at 37° C. with about 5% CO₂. The test compounds are added to media or applied directly to the air interface surface. Several exposure concentrations are included; typically 6 to 8 concentrations are employed. Following a 24 hr exposure the expression levels of several genes controlled by the ARE/EpARE promoter are monitored by RT-PCR. Cell viability is also determined for each test agent. For each chemical tested the three with the lowest variation across all exposures are pooled and used to normalize target gene expression data (FIG. 6).

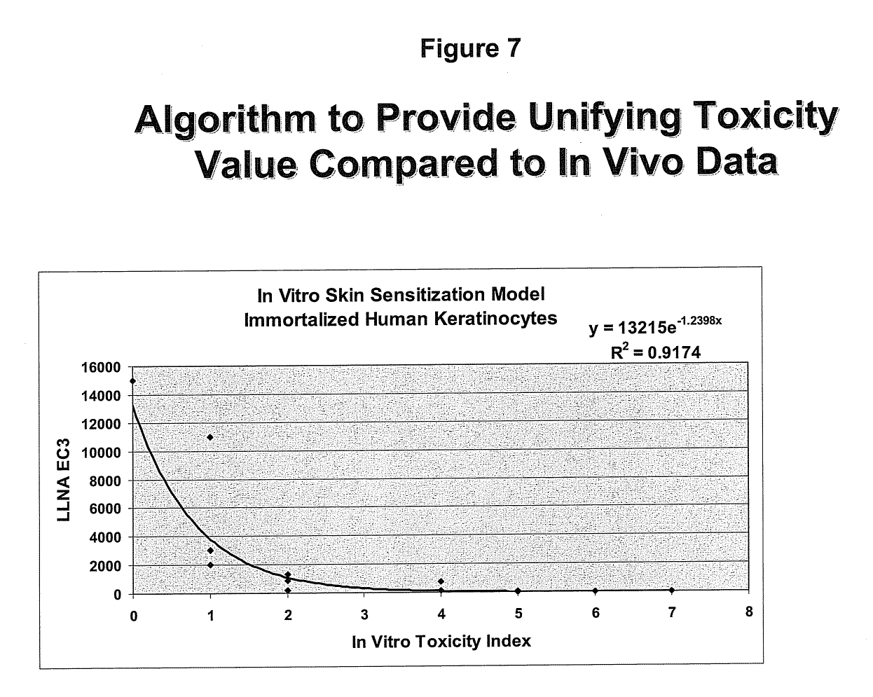

Following a 24 hr exposure, induction of the target genes in treated relative to non-treated or control groups are determined. The fold increase for each exposure concentration is placed in an input file. Cell viability is also determined and recorded for each concentration as is chemical reactivity. Concentrations that produce greater than or equal to about 50% cell death are not included in the final analysis. The IVTI is then calculated and plotted against known in vivo LLNA EC3 data and compared by exponential regression analysis (FIG. 7). The regression coefficients can then be used to calculate a predicted LLNA EC3 value for each IVTI generated.

The foregoing detailed description and drawings describe and illustrate various exemplary embodiments. The description and drawings serve to enable one skilled in the art to make and use the invention, and are not intended to limit the scope of the invention in any manner.

What is claimed is:

1. A method for predicting the in vivo skin sensitizing activity of a compound, comprising:
(a) culturing mammalian cells;
(b) applying a concentration of a test compound to the cells of step (a);
(c) measuring the expression level of one or more marker genes in the cells of step (b);
(d) optionally monitoring multiple endpoints of cell viability and general cell health;
(e) conducting a computational analysis of the concentration applied in step (b) and the expression level(s) measured in step (c); and
(f) determining a predicted in vivo sensitization value based on the analysis of step (e).

2. The method of claim 1, further comprising plotting the value calculated in step (e) against one or more known LLNA EC3 values and determining a predicted LLNA EC3 value.

3. The method of claim 1, wherein the mammalian cells are selected from the group consisting of: human keratinocytes (HaCat cells), 3D human skin cells, normal human epithelial cells (NHEK cells), MCF7 cells, H4IIE cells, and combination cultures including keratinocyte and dendritic cells.

4. The method of claim 1, wherein the test compound is applied to the cells in varying dosage amounts.

5. The method of claim 1, wherein the marker gene(s) are selected from the group consisting of: quinone reductase, IL-8, ALDHc, CYP1A, GCLA, GST, HO1, MafF, NQO1, hMTT, GAPDH CD-86, AKR, TXN, and TXN reductase.

6. The method of claim 1, wherein the in vivo sensitization value is a LLNA EC3 value.

7. The method of claim 1, wherein the in vivo sensitization value is a GPMT value.

8. The method of claim 1, wherein the in vivo sensitization value is a IVTI value.

9. The method of claim 1, further comprising performing the following steps prior to performing step (a):
(i) incubating the test compound with a polypeptide to allow for binding; and
(ii) measuring the amount of unbound polypeptide.

10. The method of claim 9, wherein the polypeptide is GSH.

11. The method of claim 9, wherein the test compound of step (i) is applied in varying dosage amounts.

12. The method of claim 9, further comprising adding human liver microsomal protein in step (i).

13. A method for determining the sensitizing activity of a compound comprising:
(a) incubating a test compound with a polypeptide to allow for binding; and
(b) measuring the amount of unbound polypeptide.

14. The method of claim 13, wherein the polypeptide is GSH.

15. The method of claim 13, wherein the test compound is applied in varying dosage amounts.

16. The method of claim 13, further comprising adding human liver microsomal protein in step (a).
